(19) United States
(12) Patent Application Publication
(10) Pub. No.: US 2024/0327329 A1
(43) Pub. Date: Oct. 3, 2024

(54) AUTOXIDATION CATALYSIS FOR CARBON CARBON BOND CLEAVAGE IN LIGNIN

(71) Applicants: Alliance for Sustainable Energy, LLC, Golden, CO (US); Wisconsin Alumni Research Foundation, Madison, WI (US)

(72) Inventors: Gregg Tyler BECKHAM, Golden, CO (US); Nina X. GU, Boston, MA (US); Chad Thomas PALUMBO, Boulder, CO (US); Kevin P. SULLIVAN, Golden, CO (US); Shannon Scot STAHL, Madison, WI (US)

(21) Appl. No.: 18/626,102

(22) Filed: Apr. 3, 2024

Related U.S. Application Data
(60) Provisional application No. 63/493,967, filed on Apr. 3, 2023.

(51) Int. Cl. C07C 67/39; C12N 1/20; C12P 7/18; C12R 1/40

(52) U.S. Cl. CPC C07C 67/39; C12N 1/205; C12P 7/18; C12R 2001/40

(57) ABSTRACT

Disclosed herein are methods and compositions of matter showing that catalytic autoxidation can be used to generate aromatic monomers from C—C linked dimers and oligomers derived from lignin. This is demonstrated by acetylating phenol-rich RCF oil and then conducting aerobic oxidation of the oligomeric fraction of poplar RCF oil with a Co/Mn/Br catalyst mixture in acetic acid. This reaction yields a collection of oxygenated aromatic monomers that represent a 17% increase in monomer yield compared to the RCF process alone.

### CROSS-REFERENCE TO RELATED APPLICATIONS

This application claims priority under 35 U.S.C. § 119 to U.S. provisional patent application No. 63/493,967 filed on 3 Apr. 2023, the contents of which are hereby incorporated in their entirety.

### CONTRACTUAL ORIGIN

The United States Government has rights in this invention under Contract No. DE-AC36-08GO28308 between the United States Department of Energy and Alliance for Sustainable Energy, LLC, the Manager and Operator of the National Renewable Energy Laboratory.

### BACKGROUND

Lignin is an abundant plant polymer rich in aromatic units and, accordingly, it has received substantial attention as a renewable alternative to petroleum-derived aromatics. Key to the valorization of lignin is an efficient method to depolymerize the polymer to its constituent aromatic monomers in high yields, but the presence of diverse recalcitrant inter-unit carbon-carbon bonds in both native and processed lignins makes this effort challenging. Most lignin depolymerization processes today solely target carbon-oxygen bonds. This work showcases an oxidative strategy to cleave carbon-carbon bonds in lignin oligomers, generating oxidized aromatic monomers that are ideal products for downstream bioconversion to valuable chemicals.

The conversion of lignin, the heterogeneous aromatic polymer found in terrestrial plant cell walls, into renewable biochemicals is critical to enable a viable lignocellulose-based bioeconomy, which would in turn aid in mitigation of the effects of anthropogenic climate change. Extensive research for lignin depolymerization has led to numerous catalytic methods to cleave the labile aryl-ether C—O bonds in lignin, whereas cleaving recalcitrant carbon-carbon bonds has received less attention. Methods and compositions of matter disclosed herein describe a method that catalytically cleaves the C—C bonds of lignin, as shown unambiguously by using lignin oils that contain only C—C linked dimers and oligomers, to produce bio-available monomers that can be further converted to a single product through a biological funneling process.

The selective conversion of lignin into renewable biochemicals is now recognized as a critical need to accelerate the commercial success of the biorefining industry. However, the inherent reactivity of lignin complicates valorization as condensation reactions during conventional biomass processing limit monomer yields and prospects of downstream depolymerization. Accordingly, recent methods for lignocellulose processing have emerged described by the lignin-first approach that preserves the structure of lignin while retaining the hemicellulose and cellulose fractions of lignocellulosic biomass for downstream upgrading. One of the most frequently reported lignin-first methods is reductive catalytic fractionation (RCF). During RCF, lignin is simultaneously fractioned and depolymerized via reductive cleavage of C—O bonds (largely beta-O-4). The low molecular weight monomers can be separated via distillation, chromatography, liquid-liquid extraction, supercritical fluid extraction, distillation, crystallization, membrane separation, and adsorption, whereas the high molecular weight fraction consists of dimeric, trimeric, and oligomeric (DTO) molecules bound through various C—C bonds and are of interest for valorization. The yields of monomers typically range 15-36 wt % of the total lignin mass in the biomass depending on the biomass source, and the monomers constitute 35-40 wt % of the extracted lignin oil. The development of methods that cleave the recalcitrant C—C bonds of the DTO of RCF lignin oil would produce monomers beyond the theoretical maximum from RCF arising from C—O bond cleavage and would make more competitive the lignin-first scheme with traditional biorefineries. In this report, we describe a catalytic method that cleaves the recalcitrant C—C bonds of the DTO fraction to obtain additional phenolic lignin monomers.

### SUMMARY

In an aspect, disclosed herein is a method for the cleavage of carbon-carbon bonds comprising 5-5, beta-1, beta-beta and beta-5 linkages in acetylated lignin oligomers produced from reductive catalytic fractionation using a Co/Mn/Br-based catalytic autoxidation system.

In an aspect disclosed herein is the use of a genetically engineered Pseudomonas sp. for a method for the cleavage of carbon-carbon bonds comprising 5-5, beta-1, beta-beta and beta-5 linkages in acetylated lignin oligomers produced from reductive catalytic fractionation using a Co/Mn/Br-based catalytic autoxidation system.

In an aspect, disclosed herein is a method for the cleavage of carbon-carbon bonds comprising 5-5, beta-1, beta-beta and beta-5 linkages in acetylated lignin oligomers produced from reductive catalytic fractionation using a Co/Mn/Br-based catalytic autoxidation system wherein the method comprises reacting the acetylated lignin oligomers produced from reductive catalytic fractionation with a mixture of a cobalt salt, a manganese salt and a bromine salt. In an embodiment, the mixture of cobalt salt, a manganese salt and a bromine salt comprises Co(OAc)2·4H2O, Mn(OAc)2·4H2O, and NaBr. In an embodiment, the mixture of a cobalt salt, a manganese salt and a bromine salt are reacted with the acetylated lignin oligomers produced from reductive catalytic fractionation at a molar ratio of three percent cobalt salt, three percent manganese salt and three percent bromine salt relative to the moles of acetylated lignin oligomers. In an embodiment, the reaction of the acetylated lignin oligomers with the mixture of a cobalt salt, a manganese salt and a bromine salt is at about 120 degrees Celsius. In an embodiment, the reaction of the acetylated lignin oligomers with the mixture of a cobalt salt, a manganese salt and a bromine salt is under about 6 bar of oxygen pressure. In an embodiment, the reaction of the acetylated lignin oligomers with the mixture of a cobalt salt, a manganese salt and a bromine salt reacts for up to 2 hours. In an embodiment, the lignin oligomers comprise vanillic acid and acetyl vanillin. In an embodiment, the carbon-carbon bonds are labile aryl-ether bonds. In an embodiment, the Co/Mn/Br-based catalytic autoxidation system comprises acetic acid. In an embodiment, the Co/Mn/Br-based catalytic autoxidation system comprises a polar solvent. In an embodiment, the Co/Mn/Br-based catalytic autoxidation system comprises a non-polar solvent. In an embodiment, the method results in up to a 17 percent increase in yield compared to a process using only reductive catalytic fractionation. In an embodiment, the method further comprises using oxygen as an oxidant. In an embodiment, in the oligomers are derived from pine lignin RCF oils. In an embodiment, the oligomers are derived from poplar lignin RCF oils. In an embodiment, the method is performed in a genetically engineered bacterium. In an embodiment, the genetically engineered bacterium is Pseudomonas sp. In an embodiment, the genetically engineered bacterium is selected from the group consisting of CJ486 or CJ781. In an embodiment, the method produces muconic acid. In an embodiment, the method produces cis, cis-muconic acid.

Other objects, advantages, and novel features of the present invention will become apparent from the following detailed description of the invention when considered in conjunction with the accompanying drawings.

### BRIEF DESCRIPTION OF THE DRAWINGS

FIG. 1 depicts the overall lignin conversion approach presented herein, featuring an oxidative C—C bond cleavage process to generate oxidized monomers that are suitable for biological funneling to a single product.

FIGS. 2A and 2B depict (A) GPC traces of acetyl RCF oil, acetyl monomer fraction, and acetyl oligomer fraction. Wt % values expressed as the weight of acetyl monomer or oligomer fraction/weight of acetyl RCF oil. (B) GPC trace of acetyl monomer fraction (top) and acetyl RCF monomer standards (bottom).

FIGS. 3A, 3B, 3C, 3D depict reaction optimizations showing total yields for the autoxidation of the acetyl oligomer substrate at variable (A) Co and Mn loadings ([Co]═[Mn]), (B) NaBr loadings, (C) temperatures, and (D) reaction times. Standard conditions for optimization utilize x=0.003 mmol, y=0.003 mmol, z=2 h, T=120° C. for 20 mg of acetyl oligomer substrate. For the standard conditions, oxidation yields are an average for four runs. Values from all other conditions are from single runs. Products were quantified by GC-FID and LC-MS.

FIG. 4 depicts monomer content in the starting acetyl oligomer material and resulting oxidation mixture (products quantified by HPLC and LC-MS). Oxidation yields are an average of four runs. Wt % values expressed as the weight of total oxidation products/weight of acetyl oligomer substrate.

FIGS. 5A, 5B, 5C depict metabolic pathway for oxidation products. FIG. 5A depicts a metabolic pathway for oxidation products from acetyl RCF oil oligomers in P. putida strain CJ781. Base treatment of the oxidation products hydrolyzed acetyl groups from aromatic monomers, yielding a mixture of syringaldehyde, syringate, vanillin, and vanillate. Syringaldehyde and syringate are converted to biomass (growth) via 3-O-methylgallate, gallate, and 4-oxalomesaconate. Vanillin and vanillate are converted to muconate via protocatechuate and catechol. FIG. 5B depicts strain CJ781 cultivated in M9 minimal medium with 1 mM/each of the model compounds syringaldehyde, syringate, vanillin, vanillate. FIG. 5C depicts strain CJ781 cultivated in M9 minimal medium with 10% v/v oxidation products from acetyl RCF oil. Gallate and catechol were not detected at significant concentrations in any of the experiments, and 4-oxalomesaconate was not measured. All cultures contained 5 mM glucose at time zero, and glucose was fed to a concentration of 5 mM every 24 h to support growth. Error bars represent the standard deviation from the mean of three biological replicates.

FIG. 6 depicts a scheme of an embodiment of methods disclosed herein.

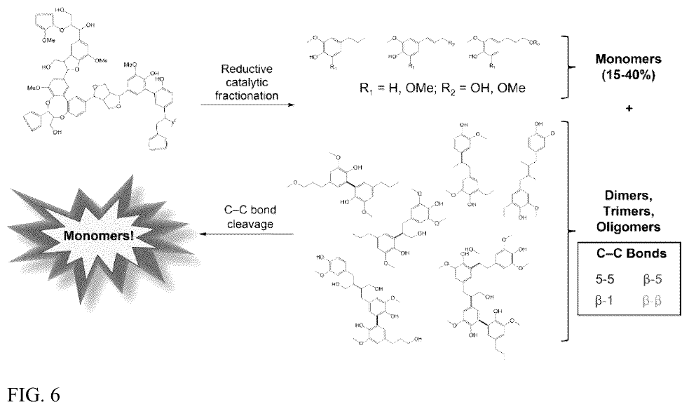

### DETAILED DESCRIPTION

Disclosed herein are methods and compositions of matter for autoxidation chemistry, mediated by metal salts (e.g., Co, Mn, Zr) and promoters (e.g., NHPI, Br) in the presence of oxygen or air with acid solvents to be able to selectively and catalytically cleave C—C bonds in lignin.

The cleavage of ether and ester bonds is a well-developed technology that can go through many routes (e.g. reductive, oxidative, etc.). However, the inherent content of C—C bonds linking aromatic monomers in lignin inherently limits the monomer yields accessible from lignin, which is key to many valorization strategies. To date, the only technology that has been reported to selectively cleave C—C bonds in lignin is not catalytic and does not yield aromatic compounds as the primary product(s).

Disclosed herein are methods and compositions of matter which describe autoxidation on phenol-protected RCF oils substrates. Further, the reaction conditions and catalyst compositions disclosed herein are in multiple embodiments.

Selective lignin depolymerization is a key step in lignin valorization to value-added products, and there are multiple catalytic methods to cleave labile aryl-ether bonds in lignin. However, the overall aromatic monomer yield is inherently limited by refractory carbon-carbon linkages, which are abundant in lignin and remain intact during most selective lignin deconstruction processes. In this work, we demonstrate that a Co/Mn/Br-based catalytic autoxidation method promotes carbon-carbon bond cleavage in acetylated lignin oligomers produced from reductive catalytic fractionation. The oxidation products include acetyl vanillic acid and acetyl vanillin, which are ideal substrates for bioconversion. Using an engineered strain of Pseudomonas putida, we demonstrate the conversion of these aromatic monomers to cis,cis-muconic acid. Overall, this study demonstrates that autoxidation enables higher yields of bio-available aromatic monomers, exceeding the limits set by ether-bond cleavage alone.

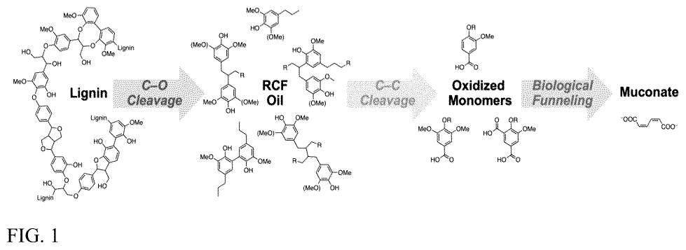

Lignin is one of the Earth's most abundant natural polymers, and it is synthesized via oxidative radical coupling reactions of monolignols that give rise to a polymer with aryl-ether bonds and several types of carbon-carbon (C—C) linkages. Lignin depolymerization to aromatic monomers is one of the most sought-after contemporary approaches to derive value from lignin. Many effective methods have been developed to this end, and near-theoretical monomer yields are now accessible, based on cleavage of beta-O-4 aryl-ether linkages. The inability of these methods to cleave refractory C—C bonds in lignin, such as those present in the 5-5, beta-1, beta-beta and beta-5 linkages, severely limits the aromatic monomer yield accessible from lignin (FIG. 1). Hardwood lignin often exhibits high beta-O-4 content and yields of aromatic monomers between 30-40 wt % are often attainable. Considerably lower yields are obtained from lignins with lower aryl-ether bond content, including those from softwoods, grasses, and extracted lignins from biorefinery and pulping processes.

Reductive catalytic fractionation (RCF) of lignin is among the most effective methods available for conversion of lignin into aromatic monomers. However, like most lignin depolymerization methods, RCF is largely limited to aryl-ether bond cleavage and generates an oligomeric byproduct that is rich in C—C linkages. Access to higher yields of aromatic monomers from lignin necessitates methods for cleavage of the C—C bonds, such as those in 5-5, beta-1, beta-beta and beta-5 linkages. Recent efforts have explored homogeneous thermal catalysis, photocatalysis, and catalytic cracking. Others have demonstrated a tandem RCF/oxidation sequence to increase monomer yields. Specifically, lignin oligomers obtained from RCF treatment of a birch feedstock were treated with a super-stoichiometric oxoammonium reagent. This reagent, which operates via a hydride transfer mechanism, led to cleavage of linkages containing C—C bonds and selectively generated 2,6-dimethoxybenzoquinone as a product in 18 wt % yield with respect to the oligomeric feedstock.

### Methods

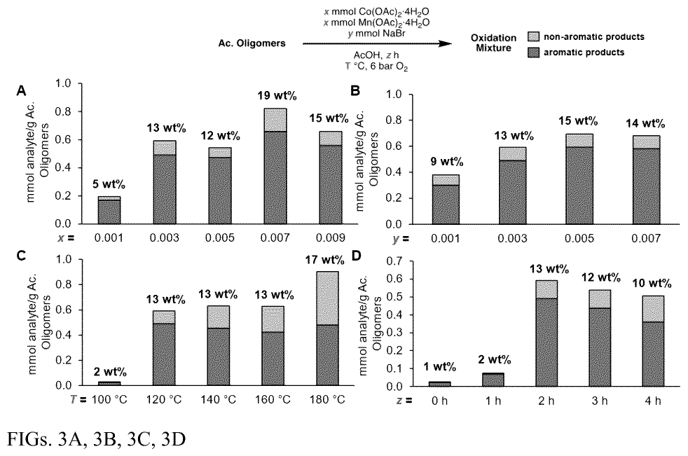

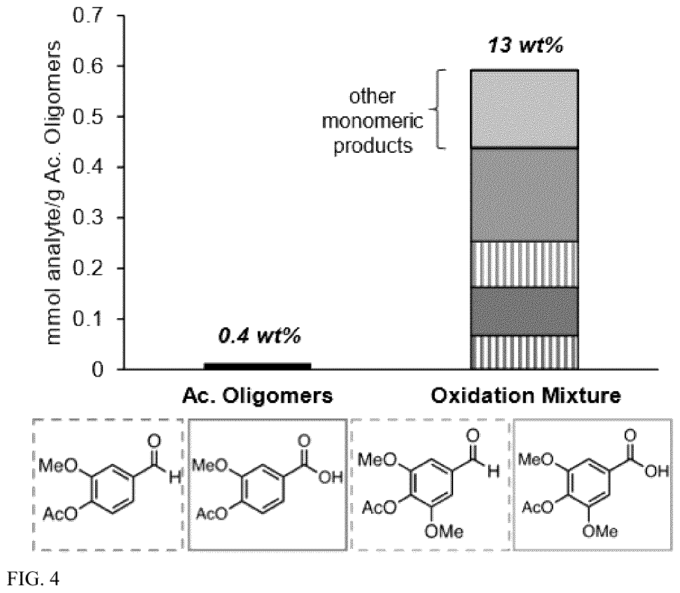

Poplar RCF oil was prepared by hydrogenolysis in methanol at 225° C. over a Ru/C catalyst. Acetylation of poplar RCF oil was accomplished by treating the poplar RCF oil with acetic anhydride and pyridine at 40° C. Vacuum distillation at 250° C. afforded an acetylated monomer distillate fraction and the acetylated oligomer fraction which remained in the distilling flask. Oxidations of the acetylated oligomer fraction were performed in 75 mL stainless steel vessels from the Parr Instrument Company. Analysis of products were conducted using GC-FID (RCF substrates, oxidation products for oligomer oxidation optimization screening in FIG. 3), HPLC (products of model compound oxidation and the optimized oligomer oxidations in FIG. 4), and LC/MS (model compound and oligomers oxidations).

### Oxidation Catalysis:

Standard oxidation of the model compounds: An acetic acid solution of substrate (0.1 mmol) was transferred to a Parr reactor containing a glass liner and teflon stir bar, and acetic acid solutions of Co(OAc)2·4H2O (0.7 mg, 0.003 mmol), Mn(OAc)2·4H2O (0.7 mg, 0.003 mmol), and NaBr (0.3 mg, 0.003 mmol) were subsequently added. An additional portion of acetic acid was added such that the total reaction volume was 15 mL. The Parr reactor was then sealed and pressurized with 29 bar air and 31 bar He at room temperature. The reaction vessels were subsequently heated at 120° C. for 2 h (reaction time not including an initial heating period of ca. 30 min), with stirring at 700 rpm. At the end of the reaction, the reactors were cooled in an ice bath and then depressurized. For product quantification, the combined weight of the Parr reactor, glass liner and stir bar was measured prior to the reaction, and the combined weight of the final reaction mixture, Parr reactor, glass liner and stir bar was measured at the end of the reaction. The final reaction mixtures were homogeneous solutions with no apparent precipitate formation. Aliquots of the reaction mixture of known mass were then analyzed by GC or LC-MS.

Standard oxidation of the acetyl oligomer fraction: An acetic acid solution of acetyl oligomer substrate (0.02 g) was transferred to a Parr reactor containing a glass liner and stir bar, and acetic acid solutions of Co(OAc)₂·4H₂O (0.7 mg, 0.003 mmol), Mn(OAc)₂·4H₂O (0.7 mg, 0.003 mmol), and NaBr (0.3 mg, 0.003 mmol) were subsequently added. An additional portion of acetic acid was added such that the total reaction volume was 15 mL. The Parr reactor was then sealed and pressurized with 29 bar air and 31 bar He at room temperature. The reaction vessels were subsequently heated to 120° C. for 2 h (reaction time not including an initial heating period of ca. 30 min), with stirring at rpm 700. At the end of the reaction, the reactors were cooled in an ice bath and then depressurized. For product quantification, the combined weight of the Parr reactor, glass liner and stir bar was measured prior to the reaction, and the combined weight of the final reaction mixture, Parr reactor, glass liner, and stir bar was measured at the end of the reaction. The final reaction mixtures were homogeneous solutions with no apparent precipitate formation. Aliquots of the reaction mixture of known mass were then analyzed by GC or HPLC.

Preparation of bacteria culture media: Oxidation reactions were performed in triplicate at 40 mg scale in scintillation vials. A portion of each sample was removed for analysis, leaving ˜30 mg of starting material per sample. Samples were dried with a nitrogen stream and stored at −20° C. until use. To prepare the substrates for bacterial cultivation, 3 mL of water and 1.2 mL of aqueous 4 M NaOH was added to each lignin sample, and mixtures were solubilized with a stir bar for 1 h at room temperature. Metal catalyst visibly precipitated and was removed by centrifuging the samples at 3000 g for 5 min. Supernatants were transferred to 25 mL flasks with stir bars and neutralized by dropwise addition of 38% HCl. Each preparation was then sterilized by passing through a 0.2 μm syringe filter. Culture media were prepared by combining 4.1 mL of sterile RCF oxidation substrate with 36.9 mL of sterile 1.1× M9 medium to achieve a final concentration of 10% v/v RCF oxidation substrate in 1× M9 medium 6.78 g/L Na₂HPO₄, 3 g/L KH₂PO₄, 0.5 g/L NaCl, 1 g/L NH₄Cl, 2 mM MgSO₄, 100 μM CaCl₂), and 18 μM FeSO₄). For control media, 40 mM stock solutions of vanillate, syringate, vanillin, and syringaldehyde were prepared by mixing each aromatic compound in water and titrating with 4 M aqueous NaOH to neutralize the solution and dissolve the compound. Aromatic stocks were then combined with 1.1× M9 medium to reach a final concentration of 1 mM each. Vanillic acid, syringaldehyde, and vanillin were obtained from Sigma-Aldrich, and syringic acid was obtained from AK Scientific.

Shake flask cultivations: Pseudomonas putida strains CJ781 and CJ486 were streaked from glycerol stocks onto LB agar (Sigma-Aldrich). The next day, biological triplicate cultures of CJ781 were prepared in 5 mL of LB medium (Sigma-Aldrich) in 15 mL glass culture tubes, using a single colony to inoculate each tube. A single culture of CJ486 was prepared in the same manner. Cultures were incubated overnight (˜16 h) at 225 rpm and 30° C. Overnight cultures were then centrifuged at 2500 g for 5 min and resuspended in 2 mL each of 1× M9 medium. For each sterile medium described above, 8 mL of medium was added to each of five 50 mL culture flasks (25 flasks in total). Sterile glucose solution was added to a concentration of 5 mM. Flasks were then inoculated with relevant overnight culture suspensions to an initial OD600 nm of 0.1. For each condition, three biological replicate flasks were prepared with CJ781, one flask was prepared with CJ486, and one flask remained uninoculated as an abiotic control. All flasks were sealed with Breathe-Easy film (Diversified Biotech) and foam caps to limit evaporation. Flasks were incubated at 225 rpm and 30° C. for 3-4 days. Every 24 h, 400 μL of culture (or abiotic control) was withdrawn from each flask. 50 μL of culture was used for OD600 nm measurement, and the remaining volume was centrifuged at 6000 g for 1 min and supernatants were passed through 0.2 μm filters for metabolite analysis. An additional 5 mM glucose was added to each biotic flask every 24 h to support growth and promote metabolite turnover.

Disclosed herein are methods and compositions of matter useful for a complementary strategy to achieve C—C cleavage in lignin that leverages catalytic autoxidation and radical reaction pathways. A cobalt/manganese/bromide co-catalyst system provides the basis for the industrial autoxidation of p-xylene to terephthalic acid, and analogous conditions have been used to support C—C cleavage in both simple hydrocarbons and synthetic plastics. Key mechanistic steps in these reactions include hydrogen atom transfer, radical trapping by O₂, and beta-scission of intermediate alkoxyl radicals. Lignin has been subjected to related conditions, but monomer yields did not exceed the monomer content of the lignin substrate. Lignin oligomers derived from RCF treatment of biomass are more amenable to such treatment and undergo successful conversion into aromatic monomers. The results disclosed herein show that a Co/Mn/Br-based catalyst system converts RCF-derived oligomers from poplar into aromatic monomers, which are then used as substrates for bioconversion to cis,cis-muconate, a precursor to bio-derived polymers (FIG. 1). By controlling the catalytic conditions, the yield of aromatic products can be maximized while limiting over-oxidation to quinone-based products that are not amenable to biological funneling to cis,cis-muconate. This pairing of catalytic aerobic oxidation that supports C—C bond cleavage and biological funneling offers a strategy to obtain higher yields of single products from lignin.

Phenol acetylation is key to enabling C—C bond cleavage via autoxidation. As an initial test of the targeted C—C bond cleavage chemistry, we explored the oxidation of model aromatic substrates (Scheme 1). Upon subjecting 4-propylguaiacol (1) to catalytic Co/Mn/Br salt mixtures (3 mol % Co(OAc)₂·4H₂O, 3 mol % Mn(OAc)₂·4H₂O, 3 mol % NaBr) with heating at 120° C. for 2 h under 6 bar O₂, we observed no C—C bond cleavage and instead recovered 1 in 87(12)% yield. This result is consistent with previous work describing the antioxidant properties of phenols and highlights the importance of phenol protection in autoxidation. In contrast, acetyl 4-propylguaiacol (2) can be converted to acetyl vanillic acid and acetyl vanillin in 47(8)% yield under the same oxidation conditions. Similarly, the oxidation of acetyl propylsyringol (3) gives a total yield of acetyl syringic acid and acetyl syringaldehyde at 42(12) %. These results on 2 and 3 demonstrate the viability of autoxidation conditions for simplified G- and S-type lignin models.

Scheme 1. Oxidation of model substrates: 4-propylguaiacol (1, top), acetyl 4-propylguaiacol (2, middle), and acetyl 4-propylsyringol (3, bottom).

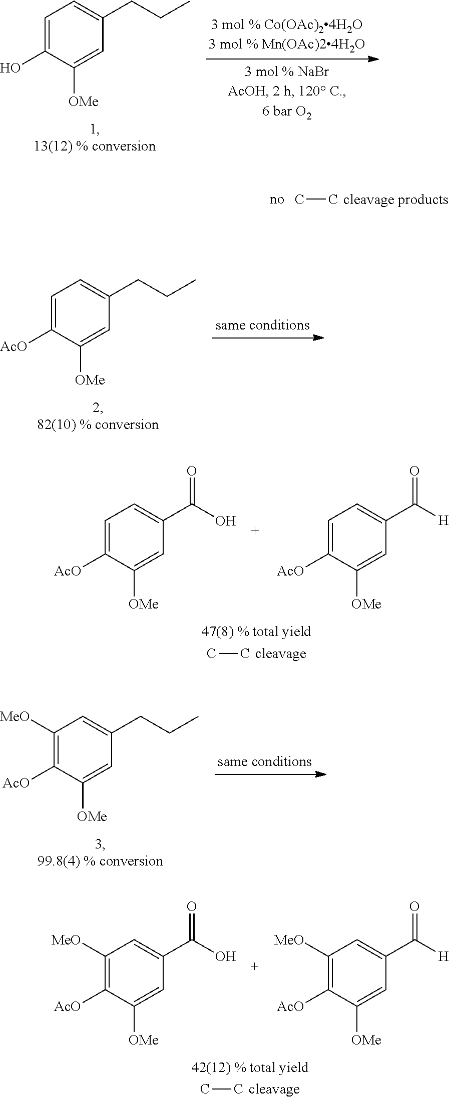

Application of autoxidation on model lignin dimers. We subsequently subjected various representative dimer models to the same autoxidation conditions and were able to detect monomeric products in most cases (Scheme 2). As a representative beta-1 dimer, diacetyl bivanillyl (4) was subjected to the same reaction conditions (3 mol % Co(OAc)₂·4H₂O, 3 mol % Mn(OAc)₂·4H₂O, 3 mol % NaBr, 120° C. for 2 h under 6 bar O₂), which afforded C—C bond cleavage products in in 64(2)% yield. The acetyl-protected beta-5 model (5) was oxidized to yield acetyl vanillic acid and the corresponding aldehyde in 8(3)% yield. Overall, the oxidations of compounds 4 and 5 demonstrate that C—C bond cleavage can be accessed on dimer models featuring common C—C linkages found in RCF oil. Specifically, cleavage of the Cbenzylic-C bonds is observed to form the corresponding benzoic acid monomer. For the 5-5 dimer model (6), full consumption of 6 was observed, but only trace products were detected.

Scheme 2. Oxidation of model dimer substrates of beta-1 (4), beta-5 (5), and 5-5 (6) linkages.

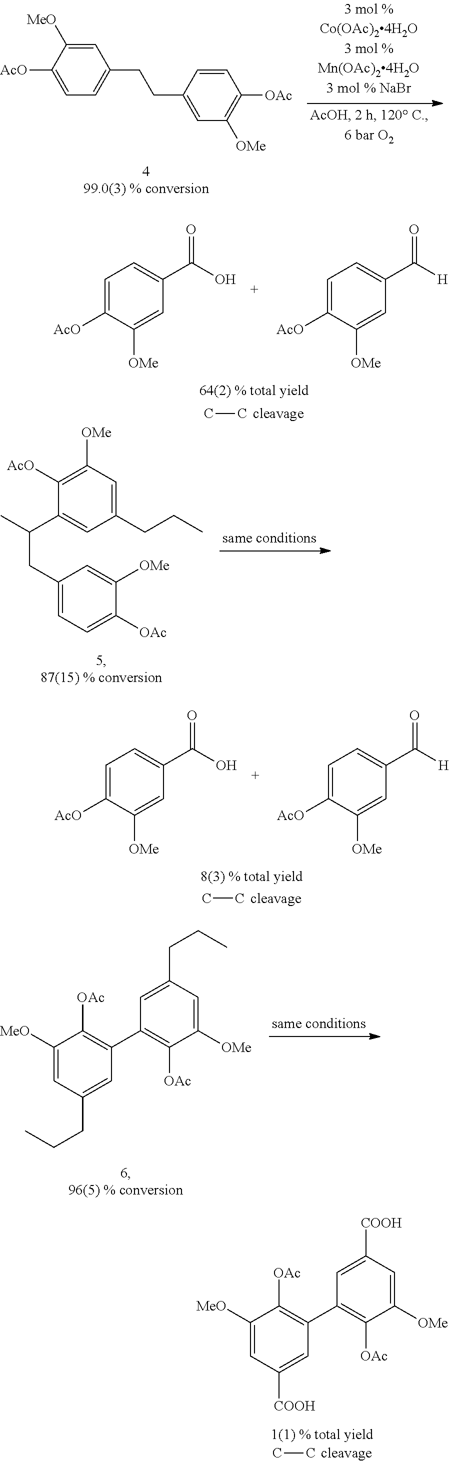

Preparation of a lignin dimer and oligomer-rich stream for autoxidation. Based on these promising model compound results, we sought to apply this autoxidation approach to a realistic lignin stream. RCF of biomass affords an “RCF oil” that contains monomers but is also rich in dimers and oligomers that exhibit 5-5, beta-1, beta-5, and beta-beta linkages. This RCF oil provides an ideal substrate to investigate the utility of Co/Mn/Br-catalytic autoxidation to supplement the yield of aromatic monomers accessible from lignin.

RCF oil was prepared by subjecting extractives-free poplar biomass to 5 wt % Ru/C under 30 bar H₂ in methanol for 6 h at 225° C. RCF monomers were quantified by GC-FID to determine a total monomer content of 1.8 mol monomer/g RCF oil. The aromatic monomer yield on a total lignin oil basis from this experiment was 34 wt %, similar to previous work. Using this oil, we functionalized free OH groups as phosphites and conducted quantitative analysis by 31P NMR spectroscopy, which yielded a phenolic content of 4.2 mmol/g RCF oil and an aliphatic OH content of 2.4 mmol/g RCF oil. With the goal of protecting the phenolic functionalities, the RCF oil was subsequently derivatized via treatment with excess acetic anhydride and pyridine at 40° C. to yield acetyl-protected RCF oil. GC-FID quantification of acetyl monomers in the acetyl RCF oil demonstrate that 78% of RCF monomers are recovered upon acetylation. GPC traces of the RCF oil and acetyl-derivatized material exhibit very similar profiles suggesting that acetylation does not alter the distribution of monomer, dimer, and oligomer fractions in the oil. Treatment of the acetylated RCF oil under the phosphite OH functionalization conditions and 31P NMR analysis confirmed the absence of phenolic and aliphatic OH groups.

Subjecting acetyl poplar RCF oil, containing the full distribution of monomers, dimers and higher molecular-weight components, to 4 wt % Co(OAc)2·4H₂O, 4 wt % Mn(OAc)₂·4H₂O, and 2 wt % NaBr at 6 bar O₂ in acetic acid yielded 21(4) wt % of monomers after heating at 120° C. for 2 h. Acetyl vanillic acid and acetyl syringic acid are the major products formed, but the quantity of oxidation monomers following oxidation (0.13 mmol/g) is lower than the initial monomer content of the starting material (1.4 mmol/g), which may be attributable to product degradation during oxidation

To gauge the stability of the oxidation products in our autoxidation conditions, we subjected acetyl vanillin and vanillic acid and acetyl syringaldehyde and syringic acid to similar conditions with 3 wt % Co(OAc)₂·4H₂O, 3 wt % Mn(OAc)₂·4H₂O, and 3 wt % NaBr. Quantification of the acid and aldehyde products by HPLC determined 75(2)% and 79(5)% of acetyl vanillin and acetyl vanillic acid, respectively, were recovered as acetyl vanillic acid. Similarly, acetyl syringaldehyde and acetyl syringic acid were recovered in 63(5)% and 92(6) % as acetyl syringic acid. These data are consistent with competing degradation of aromatic species during catalytic conditions.

Scheme 3. Product stability reactions under autoxidation conditions with mol % yields shown as value (standard deviation).

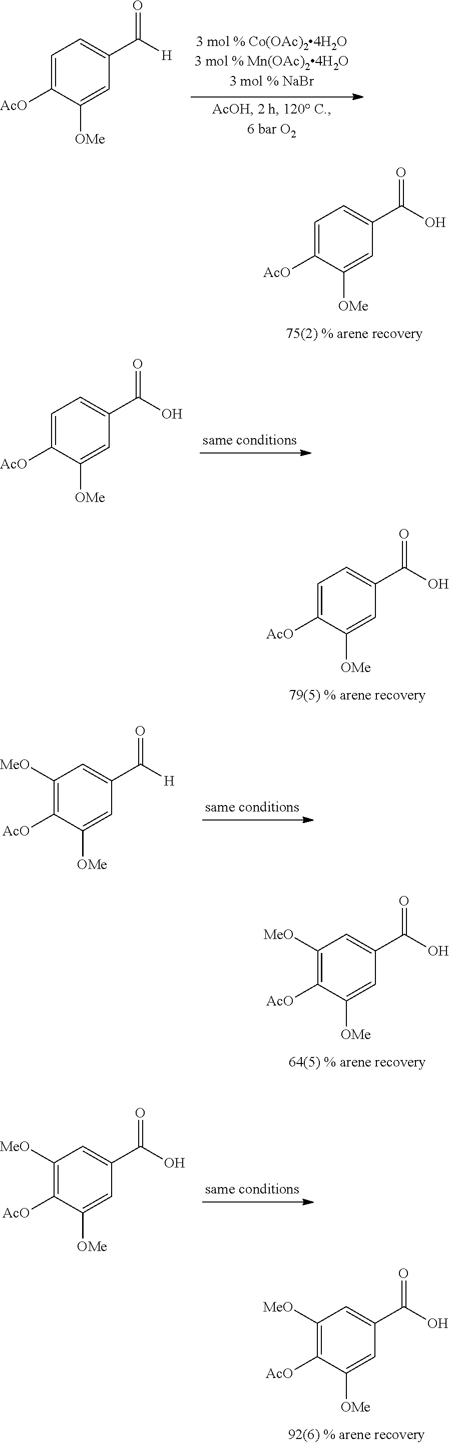

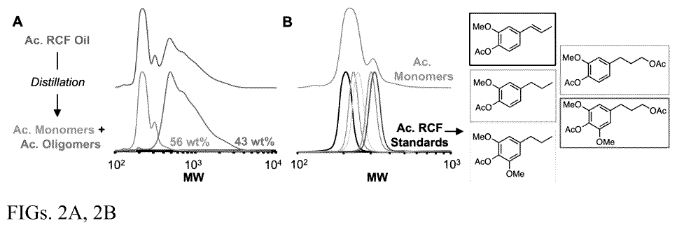

To circumvent the problem of monomer degradation, we separated the acetyl RCF oils monomers by vacuum distillation, as previously done by others and subsequently explored the oxidation of the acetyl-protected oligomers. Distillation of the acetyl RCF oil (ca. 50 mbar, 250° C.) afforded the acetyl monomer distillate as a pale-yellow oil (56 wt %), and the acetyl oligomers (43 wt %) remained in the boiling flask as a brown solid. GC-FID analysis revealed that 95% of acetyl RCF monomers were recovered in the distillate, and only trace amounts of diacetyl 4-propanolguaiacol and diacetyl 4-propanolsyringol remained the acetyl oligomer fraction. GPC analysis of the distillate (acetyl monomer fraction) confirmed the presence of only lower molecular weight species, while analysis of the acetyl oligomer fraction contains higher molecular weight compounds (FIG. 2A). Furthermore, the GPC data of the acetyl monomer fraction exhibits intensity at a very similar MW range as that of authentic standards of the main monomeric components in acetyl RCF oil, consistent with the distillate being primarily composed of acetyl RCF monomers (FIG. 2B). Phosphite functionalization and 31P NMR analysis of the acetyl monomer and acetyl dimer fractions revealed the absence of free phenolic or aliphatic OH groups, suggesting that the acetyl groups remained intact in both fractions during distillation.

Autoxidation of dimers and oligomers in RCF lignin oil. We next sought to study the effects of different reaction parameters on the oxidation of the acetyl oligomer substrate (FIG. 3). Reactions were conducted by treating the acetyl oligomer substrate with mixtures of Co(OAc)₂·4H₂O, Mn(OAc)₂·4H₂O, and NaBr, and heating the acetic acid solutions under 6 bar O₂ gave mixtures of monomers comprising aromatic and non-aromatic structures as shown in FIG. 3.

A study of Co and Mn catalyst loadings showed that increasing their loadings in a 1:1 molar ratio from 0.001 mmol to 0.003 increases the total yield of oxidation products from 5 wt % to 13 wt %. Between 0.003 and 0.009 mmol loadings, the yields range from 12-19 wt % (FIG. 3A). An increase in monomer yield was observed upon increasing NaBr loadings from 0.001 mmol (9 wt % products) to 0.003 mmol (13 wt % products), above which the monomer yields modestly varied between 13-15 wt % (FIG. 3B). Regarding the effect of reaction temperature, a large increase in yield was observed when the reaction was run at 120° C. (13 wt % products) compared to 100° C. (2 wt % products, FIG. 3C). More carboxylic acid products (acetyl syringic acid and acetyl vanillic acid) are formed compared to the corresponding aldehyde products (acetyl syringaldehyde and acetyl vanillin) when the temperature is increased from 120° C. to 140° C., but the overall yields are comparable (13 wt % products). Further increase in temperature up to 180° C. decreased the overall yield of aromatic products and increased non-aromatic products, such as 2,6-dimethoxybenzoquinone. Thus, while higher temperatures enable a moderate increase in overall monomer yield, the quantity of aromatic aldehyde and carboxylic acid products compounds suitable for downstream biological funneling decreases. Similarly, while product yields increase from 0-2 h reaction time, yields decrease at longer times (FIG. 3D), likely due to oxidative product degradation.

Based on these studies, we used 4 wt % Co(OAc)₂·4H₂O, 4 wt % Mn(OAc)₂·4H₂O, 2 wt % NaBr in acetic acid, at 120° C. for 2 h for subsequent reactions. These conditions yielded a total of 0.59 (7) mmol/g of acetylated oligomers (13 wt %) of monomeric products with acetyl vanillic acid and acetyl syringic acid being the main components identified by HPLC and LC-MS (FIG. 4). As only very small quantities of RCF monomers are present in the starting acetyl oligomer materials (0.4 wt %), the monomers generated by oxidation demonstrates the ability of these conditions to achieve C—C bond cleavage of dimers and higher molecular weight species. Overall, the average yield of additional oxidation products produced through oxidative C—C bond cleavage is 0.24 mmol/g of acetylated RCF oil. This quantity reflects a 17% increase in the acetylated monomer yield, relative to the 1.4 mmol monomers/g from the original acetylated RCF oil (eqs 1 and 2).

$$( \frac{0.59 mmol monomer}{g acetyled oligomers} ) \times ( \frac{0.4 g acetyled oligomers}{g acetylated RCF oil} ) = \frac{0.24 mmol monomers}{g acetylated RCF oil} \qquad ( 1 )$$

$$0.24 \frac{mmol monomers}{1.4 mmol monomers} \times 100 \% = 17 \% \qquad ( 2 )$$

Biological funneling of oxidation products from acetyl RCF oligomers. The monomers produced from the oxidation of poplar RCF oligomers are an ideal substrate for biological funneling, wherein heterogeneous mixtures of aromatic monomers are catabolized to a single product. Previous metabolic engineering of the aromatic catabolic bacterium Pseudomonas putida KT2440 has demonstrated the utilization of S-type monomers (syringate and syringaldehyde) as a source of carbon and energy and the conversion of G-type monomers (vanillate and vanillin) to the value-added chemical cis,cis-muconic acid. To leverage this catabolic capability, oxidation products from poplar acetyl RCF oligomers were treated with aqueous base (NaOH) to precipitate the metal catalysts and hydrolyze acetyl groups to promote bioavailability. The resulting mixture consisted of phenolic monomers and acetate, both of which can be catabolized by P. putida. The genotype of strain CJ486 is P. putida KT2440 fpvA:P_{tac}:vanAB and the genotype of CJ781 is P. putida KT2440 ΔcatRBCA::P_{tac}:catA ΔpcaHG::P_{tac}:aroY:ecdBD Δcrc ΔpobAR ΔfpvA::P_{tac}:praI:vanAB.

Engineered strains of P. putida were used to consume and convert the four major oxidation products in base-treated oxidation mixtures, namely syringate, syringaldehyde, vanillate, and vanillin. First, shake flask experiments with P. putida strain CJ486 demonstrated the ability of the strain to rapidly consume all four of these as model compounds. The same strain was then cultivated in minimal medium with 10% v/v base-treated, acetyl RCF oxidation mixtures, and all aromatic monomers were once again consumed within 24 h.

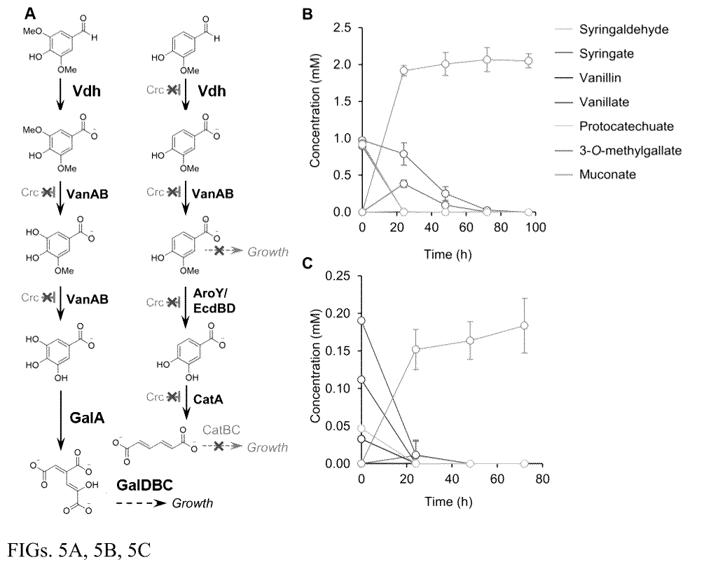

Next, P. putida strain CJ781 was employed to convert these mixtures to muconate (FIG. 5A). As expected, the model compounds syringate and syringaldehyde were consumed as sources of carbon and energy via 3-O-methylgallate, while vanillate and vanillin were converted to muconate at 100% molar yield (FIG. 5B), demonstrating viability of the engineered pathway. Similar outcomes were observed when strain CJ781 was cultivated in minimal medium with 10% v/v base-treated, oxidized acetyl RCF oil. Syringate and syringaldehyde were consumed with little to no accumulation of 3MGA, and vanillate and vanillin were converted to muconate at 100% molar yield for all replicates (FIG. 5C). Furthermore, evaporation and abiotic conversion of aromatic compounds in the RCF-derived substrates was negligible, as evidenced by a lack of compositional changes in cell-free media incubated under the same conditions.

Disclosed herein are methods and compositions of matter showing that catalytic autoxidation can be used to generate aromatic monomers from C—C linked dimers and oligomers derived from lignin. This concept is demonstrated herein by acetylating phenol-rich RCF oil and then conducting aerobic oxidation of the oligomeric fraction of poplar RCF oil with a Co/Mn/Br catalyst mixture in acetic acid. This reaction yields a collection of oxygenated aromatic monomers that represent a 17% increase in monomer yield compared to the RCF process alone.

Additionally, P. putida strains can utilize these oxidation mixtures to generate muconic acid in quantitative yield, which is a bioprivileged molecule that can be converted into performance-advantaged biopolymers and direct replacement bio-based chemicals, such as adipic acid and terephthalic acid. While these data demonstrate the viability of catalytic autoxidation to increase monomer yields from lignin, a limitation in our current experimental setup is manifested in the product degradation chemistry that is likely occurring in parallel to the productive C—C bond cleavage. Without being limited by theory, one possible pathway for arene degradation may proceed via a phenolic intermediate, as related ring-opened products have been reported in various oxidative degradation reactions of phenols, and the detected dimethoxybenzoquinone and methoxymaleic acid products may result from such a reaction. To overcome this limitation, flow chemistry could allow for improved control over the reaction conditions and residence time, thereby enabling improved selectivities and yields to aromatic products.

The foregoing disclosure has been set forth merely to illustrate the invention and is not intended to be limiting.

We claim:

1. A method for the cleavage of carbon-carbon bonds comprising 5-5, beta-1, beta-beta and beta-5 linkages in acetylated lignin oligomers produced from reductive catalytic fractionation using a Co/Mn/Br-based catalytic autoxidation system wherein the method comprises reacting the acetylated lignin oligomers produced from reductive catalytic fractionation with a mixture of a cobalt salt, a manganese salt and a bromine salt.

2. The method of claim 1 wherein the mixture of cobalt salt, a manganese salt and a bromine salt comprises Co(OAc)₂·4H₂O, Mn(OAc)₂·4H₂O, and NaBr.

3. The method of claim 2 wherein the mixture of a cobalt salt, a manganese salt and a bromine salt are reacted with the acetylated lignin oligomers produced from reductive catalytic fractionation at a molar ratio of three percent cobalt salt, three percent manganese salt and three percent bromine salt relative to the moles of acetylated lignin oligomers.

4. The method of claim 1 wherein the reaction of the acetylated lignin oligomers with the mixture of a cobalt salt, a manganese salt and a bromine salt is at about 120 degrees Celsius.

5. The method of claim 1 wherein the reaction of the acetylated lignin oligomers with the mixture of a cobalt salt, a manganese salt and a bromine salt is under about 6 bar of oxygen pressure.

6. The method of claim 1 wherein the reaction of the acetylated lignin oligomers with the mixture of a cobalt salt, a manganese salt and a bromine salt reacts for up to 2 hours.

7. The method of claim 1 wherein the lignin oligomers comprise vanillic acid and acetyl vanillin.

8. The method of claim 1 wherein the carbon-carbon bonds are labile aryl-ether bonds.

9. The method of claim 1 wherein the Co/Mn/Br-based catalytic autoxidation system comprises acetic acid.

10. The method of claim 1 wherein the Co/Mn/Br-based catalytic autoxidation system comprises a polar solvent.

11. The method of claim 1 wherein the Co/Mn/Br-based catalytic autoxidation system comprises a non-polar solvent.

12. The method of claim 1 resulting in up to a 17 percent increase in yield compared to a process using only reductive catalytic fractionation.

13. The method of claim 1 wherein the method further comprises using oxygen as an oxidant.

14. The method of claim 1 wherein the oligomers are derived from pine lignin RCF oils.

15. The method of claim 1 wherein the oligomers are derived from poplar lignin RCF oils.

16. The method of any of claim 1, wherein the method is performed in a genetically engineered bacterium.

17. The method of claim 12 wherein the genetically engineered bacterium is Pseudomonas sp.

18. The method of claim 17 wherein the genetically engineered bacterium is selected from the group consisting of CJ486 or CJ781.

19. The method of claim 12 wherein the method produces muconic acid.

20. The method of claim 12 wherein the method produces cis, cis-muconic acid.
